(19) United States
(12) Patent Application Publication
(10) Pub. No.: US 2026/0026717 A1
(43) Pub. Date: Jan. 29, 2026

(54) NON-INVASIVE METHOD AND DEVICE FOR CONTINUOUS SWEAT INDUCTION AND COLLECTION

(71) Applicant: CALIFORNIA INSTITUTE OF TECHNOLOGY, Pasadena, CA (US)

(72) Inventors: Wei GAO, PASADENA, CA (US); Yiran YANG, PASADENA, CA (US)

(73) Assignee: CALIFORNIA INSTITUTE OF TECHNOLOGY, Pasadena, CA (US)

(21) Appl. No.: 19/349,988

(22) Filed: Oct. 4, 2025

Related U.S. Application Data
(62) Division of application No. 17/993,602, filed on Nov. 23, 2022.
(60) Provisional application No. 63/283,021, filed on Nov. 24, 2021.

(51) Int. Cl. A61B 5/145; A61B 5/00; A61B 5/01; A61B 5/1477; A61B 5/16; A61B 10/00; A61N 1/32; G16H 10/20

(52) U.S. Cl. CPC A61B 5/14546; A61B 5/002; A61B 5/01; A61B 5/14521; A61B 5/1477; A61B 5/165; A61B 5/4833; A61B 5/4842; A61B 5/4845; A61B 5/4866; A61B 5/681; A61B 5/7267; A61B 5/7275; A61B 5/742; A61B 10/0064; A61N 1/325; A61B 2562/12; G16H 10/20

(57) ABSTRACT

Systems and methods for a microfluidic biosensor patch and health monitoring system may include an iontophoresis module, a multi-inlet microfluidic sweat collection and sampling module, and a molecularly imprinted polymer (MIP) organic compound sensor module. An iontophoresis module may provide for stimulation of a biofluid sample. A biofluid may be a sweat sample. Stimulation may be achieved via electrostimulation and/or application of a stimulating agent. A microfluidic sweat collection and sample module may include several adhesive layers with carefully designed inlets, channels, a reservoir, and an outlet for the efficient collection and sampling of biofluid. A MIP sensor module may quickly and accurately identify concentrations of key metabolites present in a biofluid sample which may indicate certain health conditions.

### RELATED APPLICATIONS

This application is a divisional of U.S. patent application Ser. No. 17/993,602, filed on Nov. 23, 2022, which claims the benefit of U.S. Provisional Application No. 63/283,021 filed on Nov. 24, 2021, the contents of which are incorporated herein by reference in their entirety.

### TECHNICAL FIELD

The present disclosure relates generally to systems and methods for biomarker monitoring. In particular, some implementations may relate to systems and methods for wearable biosensor monitoring of key metabolites using human sweat samples.

### BACKGROUND

Wearable bioelectronic technology offers many advantages for personalized health monitoring. Wearable devices are non-invasive and present less user error than other monitoring methods. Additionally, wearable devices offer the potential to monitor health status over time as opposed to collecting a sample that reflects health status at only a snap shot in time. This type of real-time monitoring offers more accurate and individualized diagnosis, treatment, and prevention for health conditions. Specifically wearable devices can measure pulse, respiration rate, temperature, and other health status indicators.

Sweat sensors are one type of wearable bioelectronic sensors that are particularly desirable because sweat contains many key biomarkers including electrolytes, metabolites, amino acids, hormones, and drug levels. However, existing sweat sensors face several key problems. First, existing sensors lack an effective continuous monitoring strategy. They employ only ion-selective and enzymatic electrodes and/or direct oxidation of electroactive molecules. Therefore, these sensors are only able to measure a limited set of biomarkers such as electrolytes, glucose, and lactate. These biomarkers alone do not provide a full enough picture of a human subject's health status to serve as an effective preventative tool. Additionally, these sensors often require a large sample of sweat to provide accurate analysis of biomarkers. This requires a larger and more powerful device which may not be suitable as a wearable. Therefore, monitoring and especially continuous monitoring presents a challenge due to these high power needs and the need for power storage. Existing models present additional challenges including that they require complex fabrication, are difficult to reproduce in large quantities in an affordable way, and are fragile, making them not suitable as wearable devices for long periods.

For at least these reasons, the current “gold standard” for measuring metabolites and other key biomarkers in the body is blood testing. Blood testing has several drawbacks including that it is invasive, as it requires withdrawal of blood from the veins. Also, accurate blood testing, and/or blood testing needing larger samples, generally requires a human subject to come to the lab and be tested. Because of the lab requirement and invasiveness, blood testing is generally only performed at a snapshot in time. This means that in many cases, unless a patient is experiencing a flare up or other type of health episode at the time of testing, the testing many not reveal any unusual metabolite levels until a health problem has become severe. Additionally, this episodic testing makes it extremely difficult to determine what factors may influence a patient's change in metabolite levels over time. Further, blood testing is very delicate as samples can be easily comprised by oxidation and other factors. Therefore, expensive equipment is required to process each sample, resulting in lengthy processing times and less testing overall.

Because of the lack of effective continuous monitoring strategies and high power needs, currently existing wearable health monitoring systems are unable to measure key biomarkers in a comparable way to blood testing. An effective wearable system would be highly desirable as blood testing is invasive, expensive, and offers limited health information over time.

### SUMMARY

Systems and methods are described herein related to wearable biosensors capable of continuous health monitoring. Health monitoring may include monitoring of key metabolites and may support precision nutrition and/or personalized medicine. Such a system may leverage several strategies including integration of laser-engraved graphene, redox-active nanoreporters, biomimetic “artificial antibodies,” and in situ regeneration technologies to offer several advantages. These advantages may allow for more precise monitoring over lengthier periods of time capable of more sensitive health monitoring. For example, the systems and methods disclosed herein may offer monitoring and analysis of trace-level metabolites and nutrients including all essential amino acids and vitamins. Other health conditions such as fatigue and infection, including viral infection, may be monitored.

Such a system may also leverage localized sweat simulation, microfluidic sweat sampling, and on-board signal calibration to offer additional advantages. These additional advantages may include prolonged monitoring which may continue both during states of exercise and states of rest. These additional advantages may also support a low-powered, light-weight, and low-cost wearable device which can be easily reproduced and fabricated, leading to more accessibility and greater sample sizes for machine learning operation. Advantages may also support a device that is comfortable, non-invasive, and easily worn by a human patient as needed, including for extended periods of time.

Amino acids (AAs) are organic compounds that are present in the human body and in food sources. Concentrations of amino acids may vary depending on many factors including dietary intake, genetic predisposition, gut microbiota, environmental factors, lifestyle factors including sleep and exercise, and other factors. The concentrations of amino acids present in human bodily fluids, including sweat and blood, can provide important information about the health of an individual. For example, elevated levels of branched-chain amino acids (BCAAs) including for example, leucine (Leu), isoleucine (Ile), and valine (Val) may be correlated with certain health conditions including obesity, insulin resistance, diabetes, cardiovascular disease, and pancreatic cancer. Deficiencies in amino acids, including, for example, arginine and cysteine, may indicate immune suppression and/or reduced immune-cell activation. Imbalances with other compounds, such as Tryptophan (Trp), tyrosine (Tyr) and phenylalanine (Phe), which are needed to support neurotransmitters such as serotonin, dopamine, norepinephrine, and epinephrine, may indicate neurological and/or mental health conditions. Other metabolic indicators involving, for example, Leu, Phe, and vitamin D, may be linked with severity, vulnerability, and mortality related to viral infections, including COVID-19.

Wearable sensors integrated with telemedicine could support safe and efficient monitoring of individual health which would allow for timely intervention for viral infection, including COVID-19, both for an individual and for communities.

A universal wearable biosensing strategy may combine mass-produced laser-engraved graphene (LEG), electrochemically synthesized redox-active nanoreporters (RARs), biomimetic molecularly imprinted polymer (MIP)-based ‘artificial antibodies,’ in situ regeneration and calibration technologies. Such a strategy may allow for sensitive, selective, and continuous monitoring of a wide range of trace-level biomarkers in biofluids including all nine essential AAs, and essential vitamins. Such a strategy may integrate seamlessly with prolonged iontophoresis-based on-demand sweat induction, efficient microfluidic-based sweat sampling, and in situ signal processing and wireless communication to get an autonomous health platform.

A sensor patch may be flexible and disposable and may have two iontophoresis electrodes, a multi-inlet microfluidic module, a multiplexed MIP nutrient sensor array, a temperature sensor, and an electrolyte sensor. A sensor and its electrodes may be designed based on LEG. LEG fabrication may enable large scale production, via CO₂ laser engraving, at relatively low cost. A sensor patch may also include a miniaturized module with iontophoresis control, in situ signal processing and wireless communication via Bluetooth. A sensor patch may be integrated with a mobile application for displaying, processing, and storing collected health data. A sensor patch may also be integrated into a smart watch.

Sweat may be a desirable biofluid to measure because sweat is rich in metabolites and builds up on/near the surface of skin. This makes sweat comparatively inexpensive and noninvasive to harvest and analyze versus other biofluids like blood. However, sweat composition varies highly on an individual basis and requires sensitive technology for accurate measurements. One approach may be to measure the difference between two oxidation peak heights before and after a designated period of time. For example, a small peak may be measured before a target molecule is bound to a binding site embedded in the LEG sensor. Then the difference between the small peak and a substantially higher peak measured after recognition and binding of a target molecule in an MIP template in an LEG sensor may be measured. A sensor may also be calibrated to account for temperature effects on sensitivity in real time. For instance, a reading from an LEG-based-strain-resistive temperature sensor and an ion selective Na⁺ sensor may be taken. These techniques may support accurate continuous on-body monitoring of sweat for metabolites.

Application of stimulating agents may be used for non-invasive and non-painful sweat induction. For example, carbachol/carbagel or other stimulating agents may be administered. This may offer long-term induction of sweat despite a onetime application of a small amount of carbagel. Sweat may then be collected in a multi-inlet microfluidic module. Such a module may be designed for optimal harvesting of sweat. Sweat may be repeatedly induced and sampled. The geometric design and features of the module may be selected for optimal sample efficiency. Key features may include the geometric design of the module, the number of inlets, the angle span between inlets, the orientation of inlet channels, and the flow direction into the reservoir.

Careful design of a selective binding MIP layer on an LEG may allow for sensitive and selective detection of AAs. MIPs are chemically synthesized biomimetic receptors formed by polymerizing functional monomers with template molecules. Here, a functional monomer, which may be, for example, pyrrole, and a crosslinker, which may be, for example, 3-Aminophenylboronic acid, may form a complex with a target molecule. Then, after polymerization, the functional groups of the functional monomer and crosslinker may be embedded in the polymeric structure on the LEG. Then, extraction of the target molecules may reveal binding sites on the LEG-MIP electrode that are complementary in size, shape, and charge to the target molecule. This may allow for detection without washing steps. Two detection strategies may be possible, including direct and indirect detection.

In an example embodiment, a target molecule may be detected directly. The oxidation of the target molecule in the MIP template may be able to be measured directly by differential pulse voltammetry (DPV). The difference in DPV peak current height (before and after the target binding and incubation time) may correlate directly to the analyte concentration. The direct approach may be effective for electroactive molecules. However, different electroactive molecules may be oxidized at similar potentials. Still, the approach is sufficiently selective and sensitive to distinguish between molecules. Several factors may influence sensitivity including the selected monomer, the selected crosslinker, the template ratios, the incubation periods, and other factors.

In another example embodiment, a target molecule may be detected indirectly. A RAR layer may be placed between the LEG and MIP layers. This configuration may enable rapid quantification. Target molecules may be selectively absorbed onto an imprinted polymeric layer which may decrease the exposure of the RAR to the sample after a period of binding/incubation time. Controlled-potential voltammetric techniques such as DPV or linear sweeping voltammetry may be applied to measure the RAR's oxidization peak or reduction peak. The decrease in peak current height after incubation/binding may correspond to the level of a particular analyte. In an embodiment, Prussian Blue nanoparticles (PBNPs) may make up the RAR. An indirect approach may be effective for detecting the levels of non-electroactive metabolites.

In another embodiment, methods and systems may leverage a multi-template MIP to detect levels of many different metabolites through a single sensor. In many cases, measurements for several different key metabolites are needed to form a complete health picture. For example, amino acids, vitamins, minerals, and other metabolites including glucose and uric acid, may all be desired measurements.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an example of a diagram showing a wearable sweat sensor, in accordance with various embodiments of the disclosed technology.

FIG. 2 is an example of a diagram showing an exploded diagram showing a wearable sweat sensor patch, in accordance with various embodiments of the disclosed technology.

FIG. 3A is an example of a diagram of a health monitoring system, in accordance with various embodiments of the disclosed technology.

FIG. 3B is an example of a diagram of a health monitoring system, in accordance with various embodiments of the disclosed technology.

FIG. 4 is an example of a flow diagram showing an iontophoresis method, in accordance with various embodiments of the disclosed technology.

FIG. 5 is an example of a flow diagram showing a preparation process of an LEG-MIP amino acid sensor, in accordance with various embodiments of the disclosed technology.

FIG. 6 is an example of a flow diagram showing detection methods of an LEG-MIP amino acid sensor, in accordance with various embodiments of the disclosed technology.

FIG. 7 is an example diagram of a microfluidic biofluid collection patch, in accordance with various embodiments of the disclosed technology.

FIG. 8 is an example of a diagram of a microfluidic biofluid collection patch, in accordance with various embodiments of the disclosed technology.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Wearable devices may offer highly desirable, non-invasive, and continuous monitoring of key health indicators. One type of desirable wearable is a sweat sensor. A carefully designed sweat sensor is particularly desirable because it may allow continuous, on body monitoring of key health indicators. This kind of continuous analysis may allow for personalized medical care and nutrition for an individual based on that individual's particular balance of detected metabolites. Key metabolites may include essential amino acids and vitamins. Applications for a wearable sweat sensor may include dietary nutrition intake monitoring, evaluation of stress and central fatigue, evaluation for risk of metabolic syndrome, and evaluation for risk of severe viral infection, including COVID-19.

A laser-engraved graphene (LEG) sensor may be advantageous because it may be engraved using a CO₂ laser cutter. Laser-cut wearable sensor patches may be fabricated on a large scale at a relatively low cost. This may allow for disposable sensor patches that may be worn by an individual for an extended of time, for instance twelve to twenty-four hours, and which may be replaced on a daily level. Low cost engravable, wearable, and disposable patches offer the opportunity to replace a patch daily on a human subject and collect health information over a period of several days or weeks without invasive testing and the need for a human patient to come in to a physical laboratory for repeated testing. Monitoring may occur both during periods of exercise and at rest.

### Continuous Sweat Induction and Collection

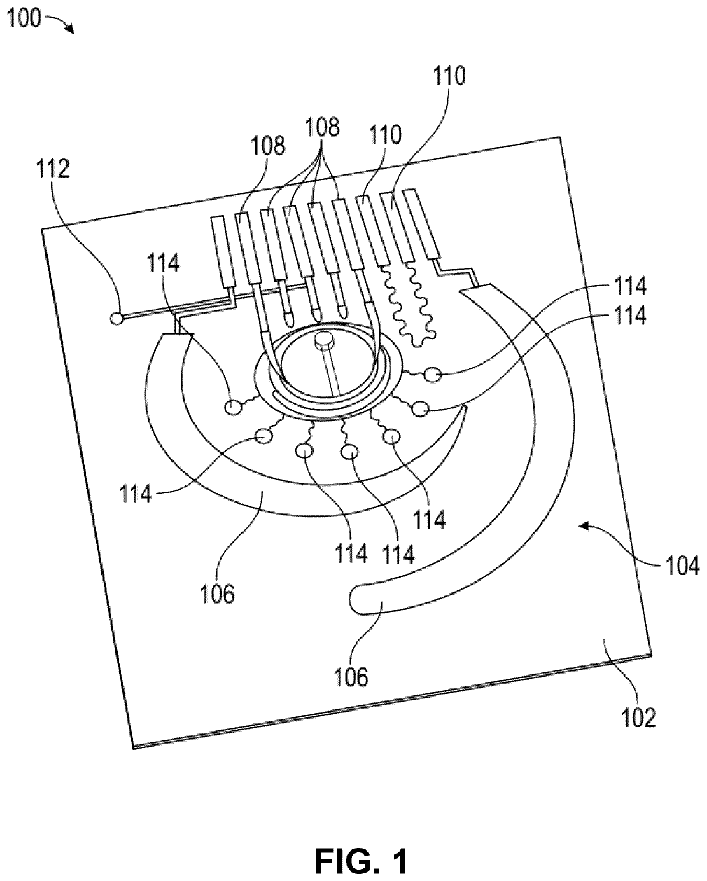

Referring now to FIG. 1, which is an example of a sweat sensor patch 100. The sweat sensor patch may include a backing layer 102. The backing layer 102 may be made of a polyimide film. The backing layer 102 may also be made of some other material. A material with lightweight, heat and chemical resistant, and flexible material may be desirable. The backing layer 102 may also include adhesive on the rear surface (not shown in FIG. 1). The adhesive may be used to attach the sensor patch directly to the skin of a human subject. The sensor patch 100 may include a biosensor array 104 on the backing layer 102. The biosensor array 104 may include several components including electrodes 106, biosensors 108, T sensors 110, an outlet 112, and inlets 114. The biosensor array 104 may be fabricated and printed onto the backing layer 102 using laser-engraved graphene (LEG) technology.

The electrodes 106 may provide a brief electrostimulation to the sweat glands of a human subject in a particular skin area. The electrostimulation may trigger the flow of sweat stimulating agents into the skin. A stimulating agent (not shown in FIG. 1) may also be added to the sweat sensor patch 100. The stimulating agent may be added in the same area as the electrodes 106, and/or may be added in the hydrogel. When the stimulating agent comes into contact with a sweat gland, via the sweat sensor patch 100, the stimulating agent may continue to stimulate the production of sweat.

The sweat sensor patch 100 may also include biosensors 108. Biosensors 108 may be configured to detect a wide variety of organic compounds present in a biofluid sample. For example, metabolites, amino acids, vitamins, minerals, hormones, antibodies, and other compounds may be detected. The biosensor 108 may be a sodium sensor. The biosensor 108 may be other sensors such as enzyme sensors, tissue-based sensors, antibody sensors, DNA sensors, optical sensors, electrochemical biosensors, piezoelectric sensors, and/or similar biosensors. A sweat sensor patch 100 may also include a T sensor 110. The T sensor 110 may be a temperature sensor. A temperature reading, in conjunction with detected concentrations of key organic compounds, may provide an indication of health status. Additionally, a temperature measurement over time, along with correlated measurements of concentrations of key organic compounds, may provide indication about changing health status or may reveal fluctuations indicative of a disease or other health condition that would not be revealed by a one-time test, such as a blood test. An electrolyte reading may indicate a patient's hydration status and/or electrolyte balance. As with a temperature measurement, an electrolyte measurement, especially over a continuous period and in conjunction with other measurements, may reveal changing health status, fluctuations indicative of disease, or a particular health condition.

The sweat sensor patch 100 may also include an outlet 112. The outlet 112 may allow for the outflow of a collected sweat sample. The outlet 112 is configured such that the outflowing sweat sample does not interfere with an incoming sweat sample. The sweat sensor patch 100 is configured to allow for collection and sample of refreshed sweat samples over an extended period of time. For example, the combination of electrode stimulation and hydrogel stimulation may induce a flow of sweat for a period of 2 to 24 hours. The sweat sensor patch 100 may also include inlets 114. Incoming sweat samples may flow through the inlets 114 and then be directed into a reservoir for collection. (The reservoir is not shown directly in FIG. 1 as the reservoir is situated below the molecularly imprinted polymer (MIP) organic compound detection module). Once the sweat sample is analyzed by the biosensors 108 and/or MIP organic compound detection module 116, the sweat sample may flow out through the outlet 112, allowing for a refreshed sample to fill the reservoir and be analyzed.

A sweat sensor patch 100 may also include a MIP organic compound detection module 116. The MIP module 116 may comprise a layer on top of the LEG layer and may be carefully designed to achieve selective binding to identify the amounts of organic compounds and/or target molecules present in a collected sweat sample. The MIP may include a functional monomer and a crosslinker. The functional monomer may be, for example, pyrrole. The crosslinker may be, for example, 3-Aminophenylboronic acid. The functional monomer and the crosslinker may form a complex with a target molecule. After polymerization, the functional groups formed by the monomer, crosslinker, and target molecule may be embedded in the LEG. The target molecule can then be extracted such that the LEG has a binding site corresponding to the target molecule in size, shape, and charge, and in this way, can detect the target molecule in the future.

In an embodiment, a sweat sensor patch 100 may include a miniature iontophoresis control module. The iontophoresis control module may allow a user to implement electrostimulation using the electrodes 106 to begin inducing a sweat flow. The electrostimulation may trigger sweat stimulating agents which may trigger the flow of sweat. The iontophoresis control module may also allow a user to implement a release of a stimulating agent to continue to induce sweat flow. The iontophoresis control module may also allow a user to set a duration for the collection of refreshed sweat samples.

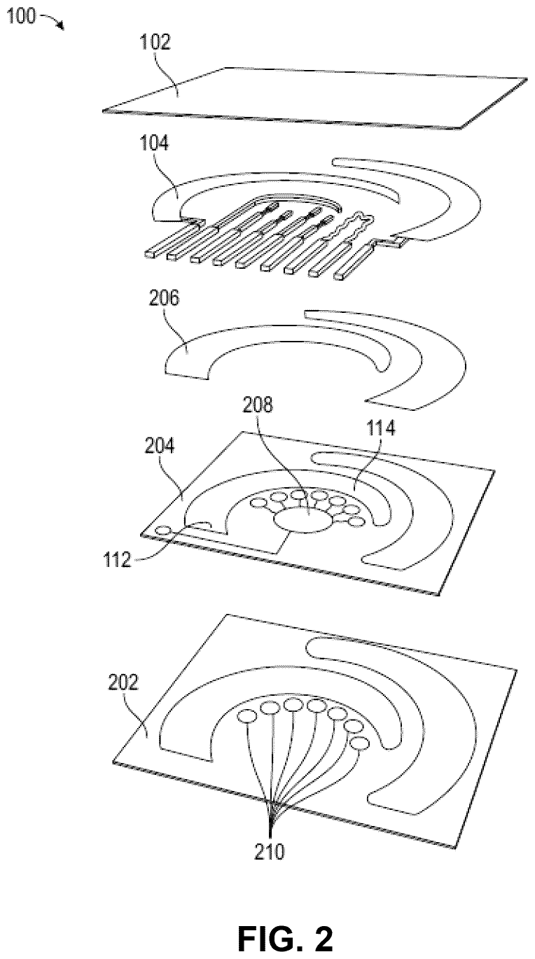

Referring now to FIG. 2, which is an example of an exploded view of the sweat sensor patch 100. The sweat sensor patch 100 may include a backing layer 102. The backing layer 102 may be made of a polyimide film. The sensor patch 100 may also include a layer having a biosensor array 104. The biosensor array 104 may be printed onto the backing layer 102 using LEG technology. The sensor patch 100 may also include a hydrogel layer 206. The hydrogel layer 206 may include a stimulating agent applied in the same area as the electrodes 106. The stimulating agent may be a carbachol gel (carbagel). The sensor patch 100 may also include a channel layer 204. The channel layer may include inlets 114, an outlet 112, and a reservoir 208. The sensor patch 100 may also include an inlet layer 202. The inlet layer may include inlets 210.

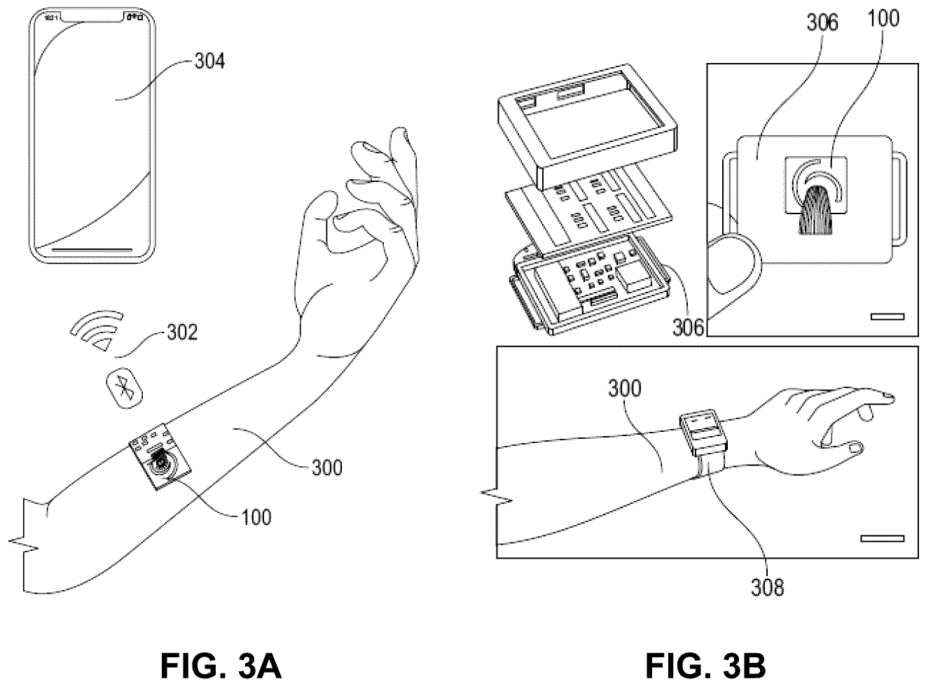

Referring now to FIG. 3A, which is an example of a sweat sensor system. The sweat sensor system may include a sweat sensor patch 100. The sweat sensor patch may be applied to a skin area 300 of a human patient. The sweat sensor patch 100 may be configured for wireless communication 302 with a mobile device 304. Wireless communication may occur via Wi-Fi, via Bluetooth or via any other wireless communication methods. The mobile device 304 may be a smart phone or other wireless device, such as a tablet, equipped with an application. The application may display detected health information from the sensor patch 100. The application may also be used to analyze and/or organize collected health data from the sensor patch 100.

Referring now to FIG. 3B, another example of a sweat sensor system is shown. The sweat sensor system may include a sweat sensor patch 100. The sweat sensor patch may be situated on a sweat sensor patch layer 306. The sweat sensor patch layer 306 may be integrated into a smartwatch device 308. The smartwatch device 308 may be worn by a human patient such that the sweat sensor patch 100 contacts a skin area 300 of the human patient. The smartwatch device 308 may communicate directly with the sweat sensor system through a wired interface. The smartwatch device 308 may also communicate with the sweat sensor system through wireless communication, including over Wi-Fi and Bluetooth. The smartwatch device 308 may display health information collected from the sweat sensor patch 100. The smartwatch device 308 may also be used to analyze and/or organize collected health data from the sweat sensor patch 100. The smartwatch device 308 may further wirelessly communicate with a mobile device.

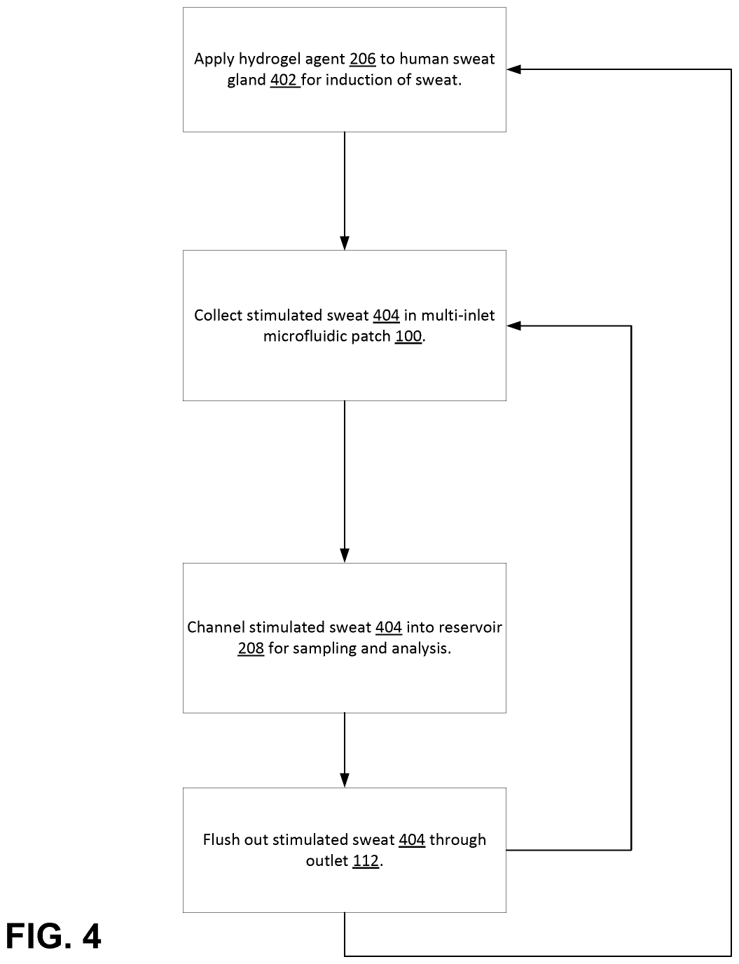

Referring now to FIG. 4, which depicts an example of a flow diagram showing a method for sweat induction and collection. First, a stimulating agent 206 is applied to a human sweat gland 402 to induce a flow of sweat. The stimulating agent 206 may be carbagel or other agents that stimulate the flow of sweat. Next, the stimulated sweat 404 is collected in a multi-inlet microfluidic sweat sensor patch (for example, the sweat sensor patch 100 of FIGS. 1-3). The induced sweat flows in through the inlets (for example, inlets 114 of FIGS. 1-2). Next, the induced sweat sample 404 is channeled from an inlet to the reservoir 208. Once in the reservoir 208, the sweat sample can be analyzed. After the sweat sample 404 is analyzed, the sweat sample 404 can be flushed out through an outlet 112. The reservoir 208 is now ready to accept a new, refreshed sweat sample. The stimulating agent 206 may support a continuous flow of sweat over a period of time. A refreshed sample can be collected without re-application of a stimulating agent 206 for a period of time. A period of time may be from within two hours up to a full, twenty-four hour day. Refreshed samples may be continuously collected in the multi-inlet microfluidic patch, channeled into the reservoir 208, analyzed, and then flushed out through the outlet 112. After a full day, a new sweat sensor patch 100 with new stimulating agent 206 may be applied and the process shown in FIG. 4 may be repeated. The process may be repeated on a daily basis for an extended period of several days, weeks, or even months. The process may also be resumed after a break of a period of minutes, hours, days, weeks, or months, to evaluate a change in a medical condition.

A sweat sensor patch and sweat sensor system, as described in reference to FIGS. 1-3, above, may measure concentrations of many different molecules and/or organic compounds. In one embodiment, a sweat sensor system may measure the concentrations of all or any of the nine essential amino acids. Amino acids are organic compounds that are present in the human body and in food sources. Concentrations of amino acids may vary depending on many factors including dietary intake, genetic predisposition, gut microbiota, environmental factors, lifestyle factors including sleep and exercise, and other factors. The concentrations of amino acids present in human bodily fluids, including sweat and blood, can provide important information about the health of an individual. For example, elevated levels of branched-chain amino acids (BCAAs) including for example, leucine (Leu), isoleucine (Ile), and valine (Val) may be correlated with certain health conditions including obesity, insulin resistance, diabetes, cardiovascular disease, and pancreatic cancer. Deficiencies in amino acids, including, for example, arginine and cysteine, may indicate immune suppression and/or reduced immune-cell activation

In another embodiment, a sweat sensor may measure concentrations of amino acids in addition to other organic compounds, including vitamins and minerals. For example, imbalances with tryptophan (Trp), tyrosine (Tyr) and phenylalanine (Phe), which are needed to support neurotransmitters such as serotonin, dopamine, norepinephrine, and epinephrine, may indicate neurological and/or mental health conditions. Other metabolic indicators involving, for example, Leu, Phe, and vitamin D, may be linked with severity, vulnerability, and mortality related to viral infections including COVID-19. Other compounds, like glucose and uric acid may also be measured to determine risk of developing, and/or severity of, a particular health condition.

In another embodiment, amino acids, vitamins, and mineral concentrations may be measured to develop a personalized nutrition plan. After measurement of initial concentrations, a human patient may be advised to make dietary modifications to account for deficiencies and/or excesses of key amino acids, vitamins, and minerals. The human patients adherence to a nutritional plan and progress may be monitored continuously with the sweat sensor patch.

In another embodiment, stress and fatigue detection and evaluation may be made based on concentrations of relevant metabolites. An object model for stress and fatigue may be trained. For example, the object model may be trained with standard stress and fatigue questionnaires. Then, machine learning methods may be used to optimize detection and evaluation of stress and fatigue through metabolic analysis, using questionnaires as an object model. For example, a machine learning model may optimize which metabolites are most accurately correlated with stress and fatigue determinations. A machine learning model may further optimize the level of detected metabolites which correlate more accurately to noteworthy stress and fatigue related health conditions. A machine learning model may be leveraged to determine at which point a human patient is experiencing too much stress and fatigue to be effective in a given role.

In another embodiment a sweat sensor may detect and measure drug compounds present in the sweat sample. Drug compounds may be measured to assess compliance with a drug treatment regimen. Drug compounds may also be measured to assess successful metabolization of a treatment drug. Drug compounds may also be measured to determine the risk and/or severity of drug toxicity due to a drug treatment regimen.

In another embodiment, the sweat sensor patch may measure the concentration of certain hormones. In another embodiment, the sweat sensor patch may measure the concentration of antibodies present in a human patient which may indicate an infection, the degree of immune response to a viral, bacterial, or fungal agent, an autoimmune disease, or another health condition.

A sweat sensor patch 100 may employ various power sources. For example, in one embodiment, a sweat sensor patch may be equipped with a lightweight battery. In another embodiment, the sweat sensor patch may be wired to a smartwatch device's power supply. In another embodiment, the sweat sensor patch may leverage a biofluid powering system to power the device with the collected sweat flow itself. In another embodiment, the sweat sensor patch may be powered with a small solar panel. In another embodiment, the sweat sensor patch may be powered by human motion.

### Polymer Detection

An MIP organic compound detection module may optimize polymer detection by creating a binding site layer in an LEG-MIP electrode. Preferred monomers may be identified for target molecules which are desirable to measure. In an embodiment, the module may use machine learning to optimize polymer detection.

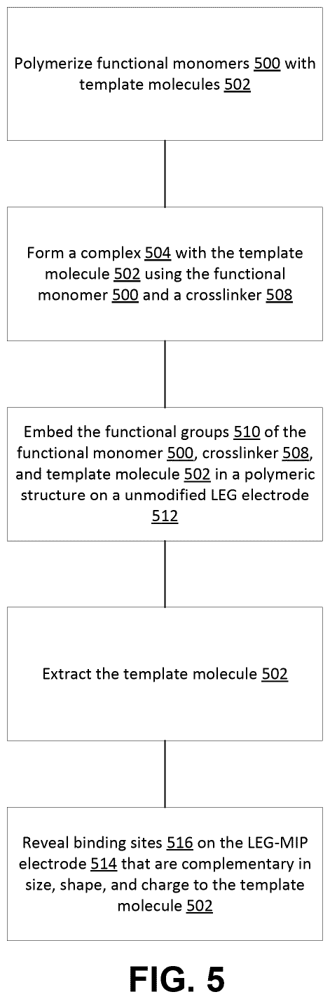

Referring now to FIG. 5, a flow diagram showing an example of a polymer detection method is shown. First, functional monomers 500 may be polymerized with template molecules 502. A preferred functional monomer 500 may be identified for each target molecule. A template molecule 502 may be a target molecule which is desirable to detect. For example, in an embodiment measuring concentrations of amino acids which may indicate the presence of metabolic syndrome, measurement of the concentration of leucine may be desirable. In that case, the template molecule may be leucine.

Next, a complex is formed using the template molecule 502, monomer 500, and crosslinker 508. The functional monomer may be, for example, pyrrole. The crosslinker may be, for example, 3-Aminophenylboronic acid. Then, after polymerization, the functional groups of the functional monomer 500, crosslinker 508, and template molecule 502 may be embedded into the polymeric structure on a pristine/unmodified LEG electrode 512 Next, the template molecule 502 may extracted. Extracting the template molecule 502 may reveal a binding site in the LEG-MIP electrode 514 that is complementary in size, shape, and charge to the template molecule 502. The LEG-MIP electrode is now equipped to detect the desired target molecule corresponding to the template molecule. The detection may be accomplished without washing steps.

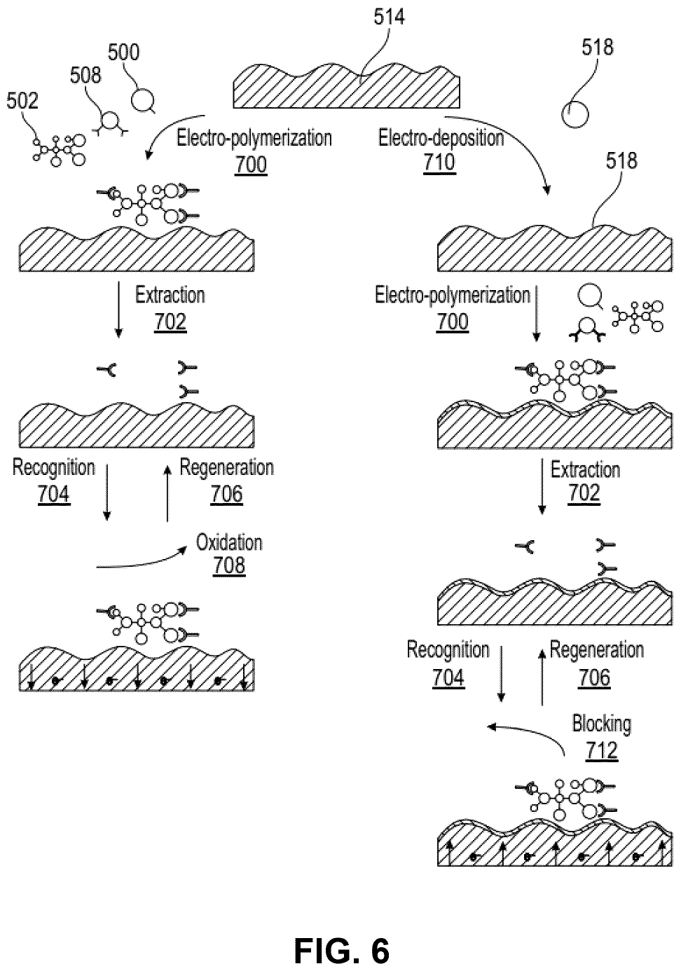

Referring now to FIG. 6, a diagram showing examples of indirect and direct detection methods for a target molecule are shown. In one embodiment, a target molecule may be detected directly after a certain period of incubation. In other words, the oxidation of the target molecule may be measured directly by differential pulse voltammetry (DPV). The difference in the DPV peak heights before and after the incubation/target binding may correlate with the analyte concentration. A direct detection approach may be effective for electroactive molecules. Different electroactive molecule may be oxidized at similar, and difficult to distinguish, potentials. The approach shown in FIG. 6 is still sufficiently selective and sensitive to distinguish between molecules, because only the target would bind to the binding sites to incur the change in the DPV peak heights. Several factors may influence the sensitivity including the selected monomer, the selected crosslinker, template ratios, incubation periods, and other factors. An object model may be trained with template molecules for optimized distinction between two template molecules having similar oxidization peaks. Machine learning techniques may then be used to optimize the selection of monomer, crosslinker, template ratio, incubation period, and other factors.

For the direct detection approach, the first step may be electro-polymerization of a monomer 500, crosslinker 508, and template molecule 502. The next step may be extraction 702 of the template molecule 502. Once the electrode is placed in a biofluid, an initial “background” scan of DPV may be performed. The next step may be a recognition 704 of target molecules in a biofluid where binding of the template/target molecules 502 occurs. After recognition 704, which occurs over a designated incubation time, oxidation 708 occurs when a second DPV is scanned and the increase between initial and the current DPV peak heights was used for target molecule quantitation. The oxidation 708 may induce the regeneration 706 to remove the bound template 512. The cycle since the initial DPV scanned can be repeated.

In another embodiment, a target molecule may be detected indirectly. An indirect detection method may include deposition of a redox-active nanoreporter (RAR) layer between LEG and MIP layers. The RAR layer may comprise, for example, Prussian blue nanoparticles. The RAR layer may enable rapid quantification. Target molecules may then be selectively absorbed into the MIP layer which may decrease exposure of the RAR layer to the sample. In such an instance, a RAR layer may experience a diminished oxidation peak in the presence of a selectively absorbed target molecule. Therefore, using a DPV technique, as above, the RAR oxidation peak height decrease (instead of increase in the direct measurement case) may correspond to a target molecule. An indirect approach may be effective for detecting the levels of non-electroactive metabolites.

Referring again to FIG. 6, an indirect detection method may first include electro-deposition 710 of a RAR layer 518 onto an LEG electrode. The next step may be electro-polymerization of a monomer 500, crosslinker 508, and template molecule 502. The next step may be extraction 702 of the template molecule 502. Once the electrode is placed in a biofluid, an initial “background” voltammetry scan may be performed as the “unblocked” RAR peak. The next step may be a recognition 704 of target molecules in a biofluid where binding of the template target molecules 502 occurs and blocking 712 of the RAR occurs. After recognition 704, which occurs over a designated incubation time, a second voltammetry scan is performed and the decrease in the peak measure at the RAR layer may correspond to the concentration of the target molecule.

### Optimization of Microfluidic Sweat Collection Patch

A microfluidic sweat collection patch may be optimized to achieve the most rapid refreshing time between samples. Several parameters may be selected for optimization. These parameters may include, for example, the placement of inlets relative to each other and a reservoir, the number of inlets, the orientation of the inlet channels, the distance between the inlets, the distance between each inlet and the reservoir, and other factors.

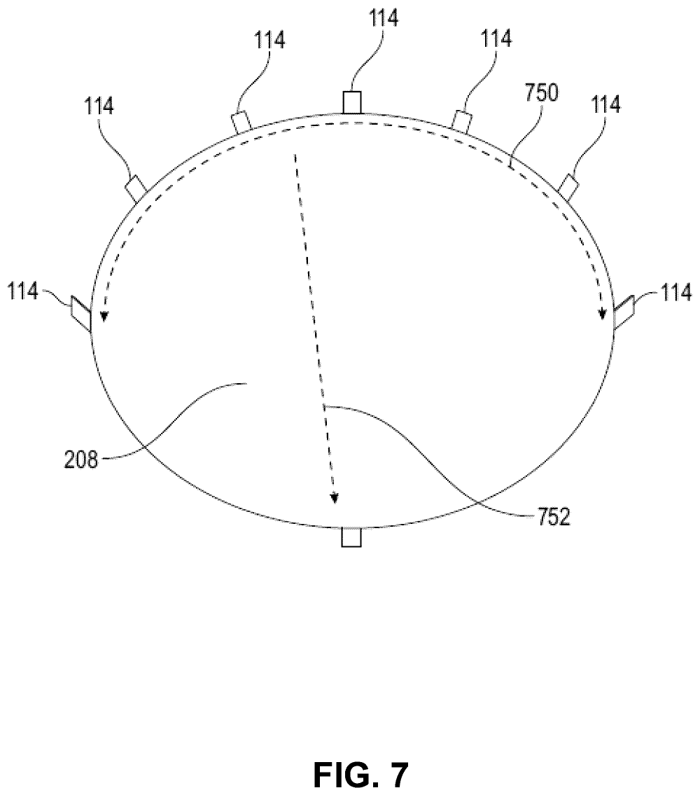

Referring now to FIG. 7, an example diagram of a microfluidic sweat collection patch is shown. The patch may include a plurality of inlets 114. The patch may also include a reservoir 208. The inlets may be configured relative to each and the reservoir at a selected angular span 750. The inlets may also be positioned to have a selected flow direction 752 relative to the reservoir 208. As shown, for example, in FIG. 7 the number of inlets may be seven. In other embodiments, there may be more or less than seven inlets. The inlets may be positioned with an angular span of 180 degrees. The inlets may be positioned to have a flow direct 752 toward the outlet.

A microfluidic sweat collection patch may also be designed to eliminate leakage of a sweat sample. For example, though electrostimulation may be applied to several neighboring sweat glands, the patch may be designed to allow for collection of a sweat sample from only gland(s) outside stimulation area while preventing leakage and hydrogel interference with the sample. This may be achieved through application of pressure on the gland the sample is taken from and through application of specialized adhesive taping of the neighboring glands and use of secure adhesive to attach the skin patch. The application of stimulating agent may also be limited to optimal parts of the patch to minimize interference.

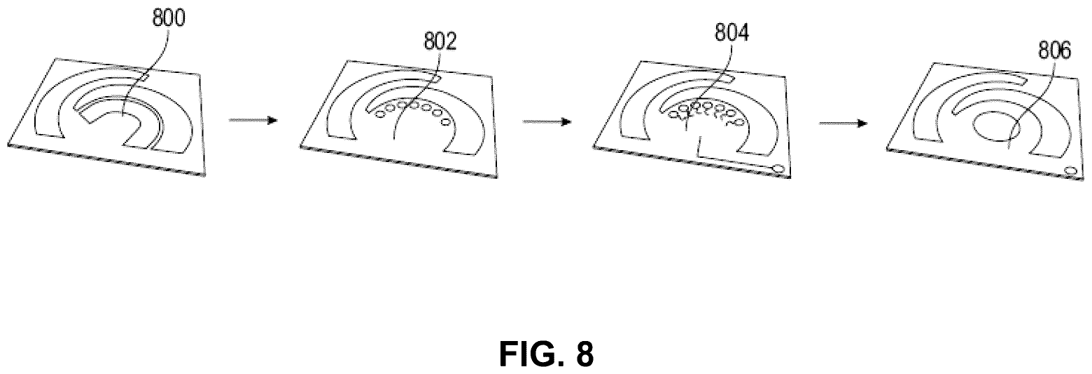

Referring now to FIG. 8, an example diagram of a microfluidic sweat collection and sampling module is shown. The module may include layers of double-sided and single-sided medical adhesives. The module may include a polyimide electrode layer. The layers of adhesives may be patterned with channels, inlets, hydrogel outlines, and reservoirs. Hydrogel outlines may be patterned to enable a flow of current from the top of the polyimide electrode layer to deliver agents into the skin. The module may include a bottom layer 800 which may be a double-sided adhesive layer in direct contact with a skin area. This bottom layer may be patterned with an accumulation well to collect sweat. The module may also include an inlet layer 802 in direct contact with the bottom accumulation layer. The inlet layer may contain a plurality of sweat inlets. The module may also include a channel layer 804 patterned with a plurality of microfluidic channels. The channel layer may be in direct contact with the inlet layer. Sweat collected in accumulation wells may flow to the inlets and then in turn flow through the channels. The module may also include a reservoir layer 806 which may be patterned with a reservoir and an outlet. Sweat may flow through the channels into the reservoir. After sampling, sweat may flow out through the outlet. The reservoir layer may lie between the channel layer and the polyimide electrode layer.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A sweat sensor patch, comprising:
an iontophoresis module, wherein the iontophoresis module administers a sweat induction agent that stimulates production of sweat;
a multi-inlet sweat sampling and collection module, wherein the multi-inlet sweat sampling and collection module collects an induced sweat sample for analysis;
a molecularly imprinted polymer (MIP) organic compound sensor module, wherein the MIP organic compound sensor module analyzes the induced sweat sample; and
a metabolite detection module, wherein the metabolite detection module identifies concentrations of target metabolites present in the collected sweat sample.

2. The sweat sensor patch of claim 1, further comprising at least one of a temperature sensor and an electrolyte sensor.

3. The sweat sensor patch of claim 1, wherein the sweat sensor patch is fabricated using laser-engraved graphene (LEG) technology.

4. The sweat sensor patch of claim 1, further comprising a miniaturized iontophoresis control module.

5. The sweat sensor patch of claim 1, further comprising an in situ signal processing and wireless communication module.

6. The sweat sensor patch of claim 5, wherein the wireless communication module communicates via Bluetooth.

7. The sweat sensor patch of claim 1, further comprising adhesive backing for direct application to skin.

8. The sweat sensor patch of claim 1, wherein the sweat sensor patch is configured to wirelessly communicate with a device, wherein the device displays collected health information.

9. The sweat sensor patch of claim 8, wherein the device is a wearable smart watch device with the iontophoresis module, the multi-inlet sweat sampling and collection module, the MIP organic compound sensor module, and the metabolite detection module comprised therein.

10. The sweat sensor patch of claim 8, wherein the device is a mobile device equipped with a mobile application for displaying, processing, and storing collected health information.

11. The sweat sensor patch of claim 1, wherein the target metabolites are selected from the group consisting of: histidine, isoleucine, leucine, lysine, methionine, phenylalanine, threonine, tryptophan, and valine.

12. The sweat sensor patch of claim 1, wherein the target metabolite comprises at least one of essential vitamins and minerals, hormones, glucose, and uric acid.

13. The sweat sensor patch of claim 1, further comprising a synthetic skin wearable disposable laboratory comprising:
a laser-engraved graphene (LEG) sensor patch;
a laser-engraved multi-inlet microfluidic sweat sampling and collection module integrated within the sensor patch; and
a laser-engraved graphene (LEG) MIP metabolite detection module integrated within the sensor patch.
